(19) United States
(12) Patent Application Publication
(10) Pub. No.: US 2018/0330822 A1
(43) Pub. Date: Nov. 15, 2018

(54) MEDICAL INFORMATION PROCESSING DEVICE AND MEDICAL INFORMATION PROCESSING METHOD

(71) Applicant: Canon Medical Systems Corporation, Otawara-shi (JP)

(72) Inventors: Kazumasa Noro, Shioyagun (JP); Yusuke Kano, Nasushiobara (JP); Longxun Piao, Nasushiobara (JP)

(73) Assignee: Canon Medical Systems Corporation, Otawara-shi (JP)

(21) Appl. No.: 15/956,398

(22) Filed: Apr. 18, 2018

(30) Foreign Application Priority Data
Apr. 18, 2017 (JP) 2017-082177

(51) Int. Cl. G16H 50/20; A61B 5/00; G16H 50/70; G16H 10/60; G16H 80/00

(52) U.S. Cl. CPC G16H 50/20; A61B 5/743; G16H 15/00; G16H 10/60; G16H 80/00; G16H 50/70

(57) ABSTRACT

A medical information processing apparatus according to an embodiment includes a processing circuitry. The processing circuitry is configured to obtain data related to health care actions and data related to symptoms of a subject occurring from the health care actions. The processing circuitry is configured to identify a health care action relevant to a health care action causing a symptom of the subject, on a basis of the data related to the health care actions and the data related to the symptoms.

### CROSS-REFERENCE TO RELATED APPLICATIONS

This application is based upon and claims the benefit of priority from Japanese Patent Application No. 2017-082177, filed on Apr. 18, 2017; the entire contents of which are incorporated herein by reference.

### FIELD

Embodiments described herein related generally to a medical information processing apparatus and a medical information processing method.

### BACKGROUND

Conventionally, for the purpose of improving quality of medical practice, hospitals and the like have introduced clinical pathways each defining a standard plan for medical consultations and treatments (which hereinafter will collectively be referred to as “health care”). As a technique for improving such clinical pathways, a method is known by which improvement items for the clinical pathways are extracted by acquiring variances indicating differences between each of the standard plans of health care written in the clinical pathways and actual health care and further analyzing the causes thereof.

### BRIEF DESCRIPTION OF THE DRAWINGS

FIG. 1 is a diagram illustrating an exemplary configuration of a medical information processing apparatus according to a first embodiment;

FIG. 2 is a table illustrating an example of clinical pathway data obtained by an obtaining function according to the first embodiment;

FIG. 3 is a table illustrating an example of patient data obtained by the obtaining function according to the first embodiment;

FIG. 4 is a table illustrating an example of actual performance data obtained by the obtaining function according to the first embodiment;

FIG. 5 is a table illustrating an example of variance data obtained by the obtaining function according to the first embodiment;

FIG. 6 is a table illustrating an example of variance code master data obtained by the obtaining function according to the first embodiment;

FIG. 7 is a table illustrating an example of correlation rule data generated by an extracting function according to the first embodiment;

FIG. 8 is a drawing illustrating an example of a relevant cause identifying process performed by an identifying function according to the first embodiment;

FIG. 9 is a table illustrating an example of execution item master data used by the identifying function according to the first embodiment;

FIG. 10 is a drawing illustrating another example of the execution item master data used by the identifying function according to the first embodiment;

FIG. 11 is a table illustrating examples of relevant causes identified by the identifying function according to the first embodiment;

FIG. 12 is a table illustrating an example of an advantageous effect predicting process performed by a predicting function according to the first embodiment on candidates for an improvement plan related to a timing change;

FIG. 13 is a table illustrating another example of the advantageous effect predicting process performed by the predicting function according to the first embodiment on the candidates for the improvement plan related to the timing change;

FIG. 14 is a table illustrating an example of an advantageous effect predicting process performed by the predicting function according to the first embodiment on candidates for an improvement plan related to a type change;

FIG. 15 is a table illustrating another example of the advantageous effect predicting process performed by the predicting function according to the first embodiment on the candidates for the improvement plan related to the type change;

FIG. 16 is a table illustrating an example of an advantageous effect predicting process performed by the predicting function according to the first embodiment on candidates for an improvement plan related to a change between execution/non-execution;

FIG. 17 is a table illustrating another example of the advantageous effect predicting process performed by the predicting function according to the first embodiment on the candidates for the improvement plan related to the change between execution/non-execution;

FIG. 18 is a drawing illustrating an example of a screen displayed by a display controlling function according to the first embodiment;

FIG. 19 is a flowchart illustrating a processing procedure in a process performed by the medical information processing apparatus according to the first embodiment;

FIG. 20 is a drawing illustrating an example of a relevant cause identifying process performed by an identifying function according to a second embodiment;

FIG. 21 is a table illustrating an example of cost data used by a predicting function according to a third embodiment; and

FIG. 22 is a table illustrating an example of an advantageous effect predicting process performed by the predicting function according to the third embodiment on candidates for an improvement plan.

### DETAILED DESCRIPTION

A medical information processing apparatus according to an embodiment includes an obtaining unit and an identifying unit. The obtaining unit is configured to obtain data related to health care actions and data related to a subject occurring from the health care actions. The identifying unit is configured to identify a health care action relevant to a health care action causing a symptom of the subject, on a basis of the data related to the health care actions and the data related to the symptoms.

In the following sections, exemplary embodiments of a medical information processing apparatus and a medical information processing method will be explained in detail, with reference to the accompanying drawings.

### First Embodiment

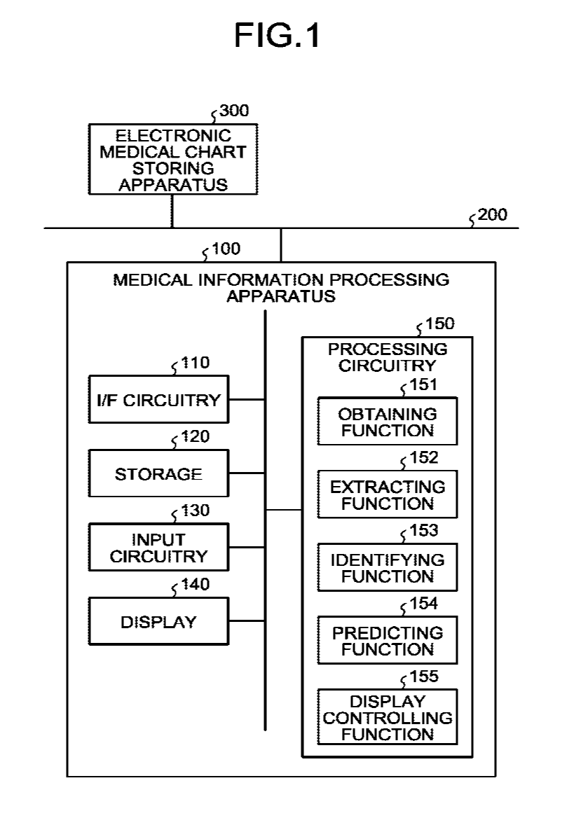

FIG. 1 is a diagram illustrating an exemplary configuration of a medical information processing apparatus according to a first embodiment.

For example, as illustrated in FIG. 1, a medical information processing apparatus 100 according to the first embodiment is connected to an electronic medical chart storing apparatus 300 via a network 200 so as to be able to communicate therewith. For example, the medical information processing apparatus 100 and the electronic medical chart storing apparatus 300 are installed in a hospital or the like and are connected to each other via the network 200 realized with an intra-hospital Local Area Network (LAN) or the like.

The electronic medical chart storing apparatus 300 is configured to store therein health care data related to various types of health care provided at the hospital or the like. For example, the electronic medical chart storing apparatus 300 is installed as a part of an electronic medical chart system introduced at the hospital or the like and is configured to store therein the health care data generated by the electronic medical chart system. For example, the electronic medical chart storing apparatus 300 is realized by using a computer device such as a database (DB) server or the like and is configured to store the health care data into a semiconductor memory element such as a Random Access Memory (RAM), a flash memory, or the like, or a storage such as a hard disk or an optical disk.

The medical information processing apparatus 100 is configured to obtain health care data from the electronic medical chart storing apparatus 300 via the network 200 and to perform various types of information processing processes by using the obtained health care data. For example, the medical information processing apparatus 100 is realized by using a computer device such as a workstation.

More specifically, the medical information processing apparatus 100 includes interface (I/F) circuitry 110, a storage 120, input circuitry 130, a display 140, and processing circuitry 150.

The I/F circuitry 110 is connected to the processing circuitry 150 and is configured to control transfer of various types of data and communication performed with the electronic medical chart storing apparatus 300. For example, the I/F circuitry 110 is configured to receive health care data from the electronic medical chart storing apparatus 300 and to output the received health care data to the processing circuitry 150. For example, the I/F circuitry 110 is realized by using a network card, a network adaptor, a Network Interface Controller (NIC), or the like.

The storage 120 is connected to the processing circuitry 150 and is configured to store therein various types of data. For example, the storage 120 is configured to store therein the health care data received from the electronic medical chart storing apparatus 300. For example, the storage 120 is realized by using a semiconductor memory element such as a Random Access memory (RAM), a flash memory, or the like, or a hard disk, an optical disk, or the like.

The input circuitry 130 is connected to the processing circuitry 150 and is configured to convert an input operation received from an operator into an electrical signal and to output the electrical signal to the processing circuitry 150. For example, the input circuitry 130 is realized by using a trackball, a switch button, a mouse, a keyboard, a touch panel, and/or the like.

The display 140 is connected to the processing circuitry 150 and is configured to display various types of information and various types of image data output from the processing circuitry 150. For example, the display 140 is realized by using a liquid crystal monitor, a Cathode Ray Tube (CRT) monitor, a touch panel, or the like.

The processing circuitry 150 is configured to control constituent elements of the medical information processing apparatus 100 in accordance with the input operation received from the operator via the input circuitry 130. For example, the processing circuitry 150 is configured to store the health care data output from the I/F circuit 110 into the storage 120. Further, for example, the processing circuitry 150 is configured to read the health care data from the storage 120 and to display the read health care data on the display 140. For example, the processing circuitry 150 is realized by using a processor.

An overall configuration of the medical information processing apparatus 100 according to the first embodiment has thus been explained. The medical information processing apparatus 100 according to the first embodiment configured as described above has functions for presenting an effective improvement plan related to clinical pathways introduced at the hospital or the like.

More specifically, the processing circuitry 150 includes an obtaining function 151, an extracting function 152, an identifying function 153, a predicting function 154, and a display controlling function 155. The obtaining function 151 is an example of the obtaining unit. The extracting function 152 is an example of an extracting unit. The identifying function 153 is an example of the identifying unit. The predicting function 154 is an example of a predicting unit. The display controlling function 155 is an example of a display controlling unit.

The obtaining function 151 is configured to obtain data related to health care actions and data related to symptoms of one or more patients (examined subjects) occurring from the health care actions.

In the first embodiment, an example will be explained in which the data related to the health care actions is data related to health care actions in a clinical pathway. In this regard, although clinical pathways are, generally speaking, often applied to hospitalized patients, the data related to the health care actions in the present example does not necessarily have to be data related to health care actions taken for hospitalized patients and does not necessarily have to be data related to health care actions taken according to a clinical pathway. For example, the data related to the health care actions may be data related to health care actions taken for ambulatory patients or outpatients.

Further, in the first embodiment, an example will be explained in which the data related to the symptoms is data related to variances. In this situation, the symptoms and the variances may include various types of situations that may occur as a result of adversely affecting the patient by taking a health care action.

More specifically, the obtaining function 151 obtains, from the electronic medical chart storing apparatus 300, clinical pathway data, patient data, actual performance data, variance data, and variance code master data. Further, the obtaining function 151 stores the obtained pieces of data into the storage 120.

In this situation, the clinical pathway data is data that has recorded therein, for each clinical pathway, a health care action to be taken, an outcome to be evaluated, the day on which the health care action is scheduled to be taken, and the like. The patient data is data that has recorded therein basic information of the patient. The actual performance data is data that has recorded therein a history of health care actions that have been taken for the patient as well as progress in the status of the patient, and the like. The variance data is data generated when a deviation from the clinical pathway has occurred and has recorded therein the date on which the variance occurred, a category code and/or text indicating a reason for the occurrence, and the like. The variance code master data is data that has recorded therein categories of the variances.

For example, the obtaining function 151 converts the pieces of data obtained from the electronic medical chart storing apparatus 300 into a format optimal for a clinical pathway analysis and stores the result of the conversion into the storage 120. In the present example, it is assumed that the information included in the pieces of data is directly obtained from the data stored in the electronic medical chart storing apparatus 300; however, possible embodiments are not limited to this example. For instance, when the information included in the pieces of data also contain some information that is not directly obtained from the data stored in the electronic medical chart storing apparatus 300, the obtaining function 151 may store the information into the storage 120 after converting the information while using a conversion-purpose table. In that situation, the conversion-purpose table is stored in the storage 120 in advance.

When obtaining the pieces of data, the obtaining function 151 may obtain only such data that is related to the patients to whom the clinical pathway was applied or may obtain such data that is related to both the patients to whom the clinical pathway was applied and the patients to whom the clinical pathway has not been applied.

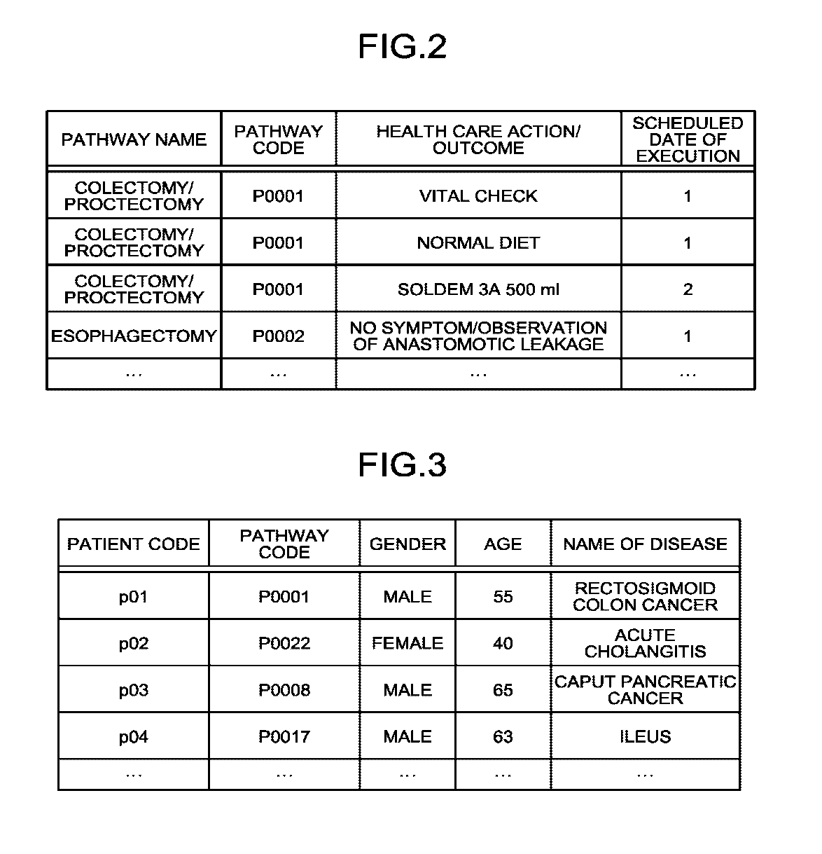

FIG. 2 is a table illustrating an example of the clinical pathway data obtained by the obtaining function 151 according to the first embodiment.

For example, as illustrated in FIG. 2, the clinical pathway data includes, as data items thereof, a pathway name, a pathway code, a health care action/outcome, and a scheduled date of execution. In this situation, as the pathway name, the name of the clinical pathway is set. Further, as the pathway code, a code uniquely identifying the clinical pathway is set. Further, as the health care action/outcome, information indicating a health care action taken according to the clinical pathway or an outcome (a goal for the patient's status to be achieved in a specific period of time) is set. For example, the information indicating the health care action may include descriptions of an observation, medication, a test, a procedures, an instruction, nutrition, an explanation, and the like that are commonly included in the clinical pathway. Further, as the scheduled date of execution, a scheduled date on which an evaluation is to be made on the health care action or the outcome is set. The scheduled date of execution may be indicated with smaller units using a time of the day.

FIG. 3 is a table illustrating an example of the patient data obtained by the obtaining function 151 according to the first embodiment.

For example, as illustrated in FIG. 3, the patient data includes, as data items thereof, a patient code, a pathway code, the gender, the age, and the name of the disease. In this situation, as the patient code, a code uniquely identifying the patient is set. Further, as the pathway code, a code uniquely identifying the clinical pathway (which is the same as the pathway code illustrated in FIG. 2) is set. As the gender, the gender of the patient is set. Further, as the name of the disease, the name of the disease of the patient is set. Besides the examples of information listed above, the patient data may include other pieces of information that have been confirmed when the application of the clinical pathway is started, such as the height, the weight, a hospitalization history, allergies, and the like of the patient.

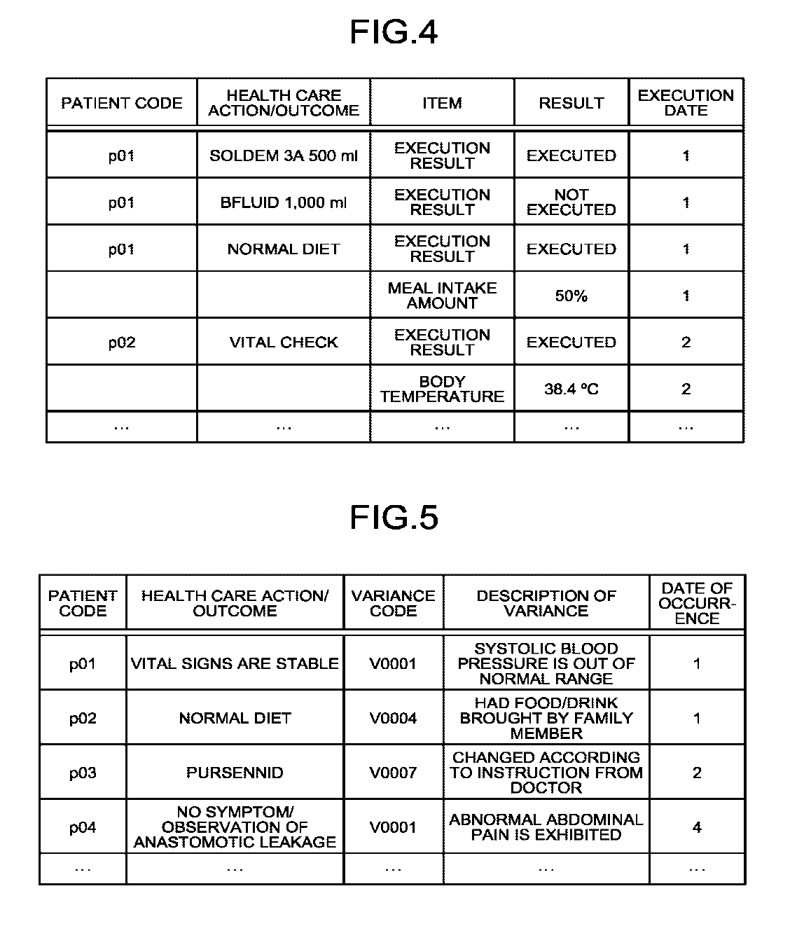

FIG. 4 is a table illustrating an example of the actual performance data obtained by the obtaining function 151 according to the first embodiment.

For example, as illustrated in FIG. 4, the actual performance data includes, as data items thereof, a patient code, a health care action/outcome, an item, a result, and an execution date. In the actual performance data, the health care action/outcome, the item, the result, and the execution date are set while being kept in association with the patient code.

In this situation, as the patient code, a code uniquely identifying the patient is set (which is the same as the patient code illustrated in FIG. 3). Further, as the health care action/outcome, information indicating either the health care action taken for the patient or an outcome thereof is set (which is the same as the health care action/outcome illustrated in FIG. 2). Further, as the item, an item obtained by evaluating the health care action or the outcome is set. Furthermore, as the result, a result obtained by evaluating the health care action or the outcome is set. As the result, data (e.g., a meal intake amount (%), the body temperature (° C.), etc.) obtained as a result of the health care action is set, in addition to an execution result (executed/not executed) of the health care action. Further, as the result, an evaluation result (achieved/not achieved) of the outcome is set. Further, as the execution date, the date on which an evaluation is made on the health care action or the outcome is set.

FIG. 5 is a table illustrating an example of the variance data obtained by the obtaining function 151 according to the first embodiment.

For example, as illustrated in FIG. 5, the variance data includes, as data items thereof, a patient code, a health care action/outcome, a variance code, a description of variance, and a date of occurrence. In this situation, in the variance data, the health care action/outcome, the variance code, the description of variance, and the date of occurrence are set while being kept in association with the patient code.

In this situation, as the patient code, a code uniquely identifying the patient is set (which is the same as the patient code illustrated in FIG. 3). Further, as the health care action/outcome, information indicating the health care action taken for the patient or an outcome thereof is set (which is the same as the health care action/outcome illustrated in FIG. 2). Further, as the variance code, a code related to a cause of the variance is set. Further, as the description of variance, information describing the variance occurring from the clinical pathway is set. For example, as the description of variance, text information describing details of the variance is set. Further, as the date of occurrence, the date on which the variance occurred is set.

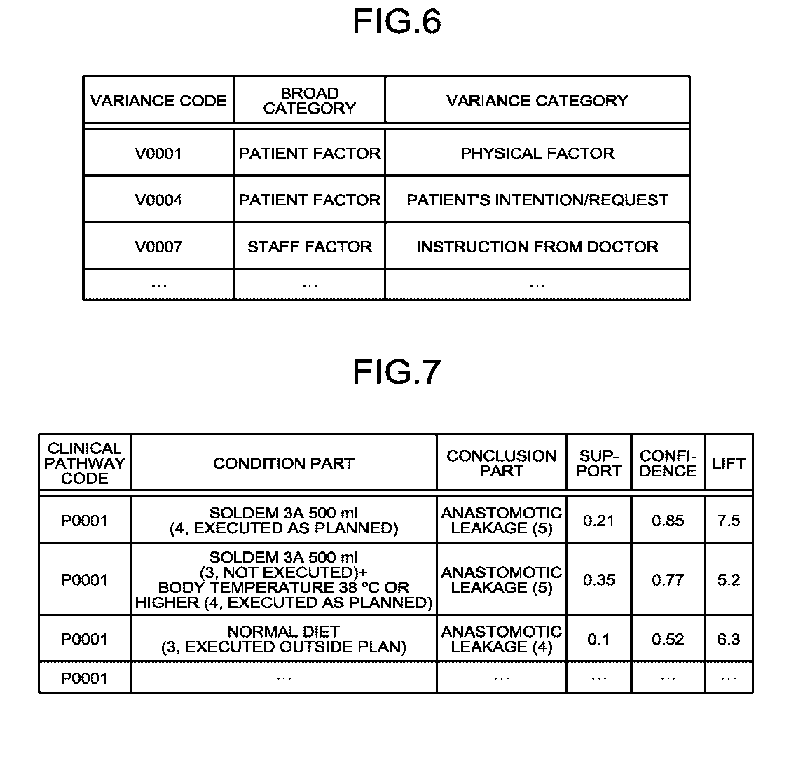

FIG. 6 is a table illustrating an example of the variance code master data obtained by the obtaining function 151 according to the first embodiment.

For example, as illustrated in FIG. 6, the variance code master data includes, as data items thereof, a variance code, a broad category, and a variance category. In this situation, as the variance code, a code related to a cause of the variance is set (which is the same as the variance code illustrated in FIG. 5). Further, as the broad category, a broad category (e.g., a patient factor, a staff factor, a facility factor, a society factor, etc.) of the cause of the variance is set. Further, as the variance category, a smaller category (e.g., a physical factor, the patient's intention or request, an instruction from the medical doctor, etc.) of the cause of the variance is set.

Returning to the description of FIG. 1, the extracting function 152 is configured to extract correlation information indicating a level of strength of correlation between a specific variance and a cause thereof, on the basis of the data related to the health care actions taken according to the clinical pathways and the data related to the variances occurring from the clinical pathways.

More specifically, as the correlation information indicating the level of strength of correlation between the specific variance and the cause thereof, the extracting function 152 extracts a correlation rule defined by a set made up of the specific variance and an element representing a cause thereof, by using information of the patient data, he actual performance data, and the variance data stored in the storage 120. In this situation, as a method for generating the correlation rule, it is acceptable to use any of various types of publicly-known analyzing methods.

In the first embodiment, the extracting function 152 generates the correlation rule by using an association analysis, on the assumption that it is possible to obtain a plurality of sets each made up of a correlation rule and a numerical value expressing the level of strength of the correlation. Alternatively, the extracting function 152 may use either a time-series association analysis or a sequential pattern mining scheme each of which is an association analysis taking the order of occurrence into consideration.

The association analysis is to extract a rule “When the condition X is satisfied, Y occurs”, where an item serving as a condition part is defined as X, while an item serving as a conclusion part is defined as Y. Generally speaking, the rule is evaluated while using support, confidence, and lift defined as indicated below, as index values.

$$\begin{matrix}Support ( X ⇒ Y ) = \frac{n ( X ⋂ Y )}{n ( A )} & ( 1 ) \\ Confidence ( X ⇒ Y ) = \frac{n ( X ⋂ Y )}{n ( X )} & ( 2 ) \\ Lift ( X ⇒ Y ) = \frac{Confidence ( X ⇒ Y )}{n ( Y ) / n ( A )} & ( 3 )\end{matrix}$$

In the expressions above, n(X) denotes the number of transactions that each include X, whereas n(Y) denotes the number of transactions that each include Y. Further, n(X∩Y) denotes the number of transactions that each include both X and Y, whereas n(A) denotes the total number of transactions.

In the first embodiment, the extracting function 152 performs the association analysis while using, as the transactions, a set made up of data related to health care actions/outcomes that occurred from the start to the end of a clinical pathway, data related to variances that occurred from the start to the end of the clinical pathway, and data related to the patient to whom the clinical pathway was applied.

More specifically, the extracting function 152 receives an operation to designate a clinical pathway and a variance from the operator via the input circuitry 130. After that, by referring to the patient data, the extracting function 152 identifies data related to one or more patients to whom the clinical pathway designated by the operator was applied. Further, by referring to the actual performance data, the extracting function 152 identifies, for each of the identified patients, data related to either a health care action taken for the patient or an outcome thereof. Further, by referring to the variance data, the extracting function 152 identifies, for each of the identified patients, data related to a variance occurring from the health care action taken for the patient. After that, the extracting function 152 generates, as a transaction, a set made up of the corresponding data related to the health care action/outcome, the corresponding data related to the variance, and the corresponding data related to the patient.

In this situation, because items used in association analyses are required to be qualitative data, data having numerical value data is converted into qualitative data. For example, the items are each converted into a label on a nominal scale, such as “Soldem 3A 500 ml (1, executed as planned)” when Soldem 3A 500 ml was administered on day 1 as planned in a clinical pathway, “Soldem 3A 500 ml (1, not executed)” when Soldem 3A 500 ml was not administered as planned, or “Bfluid 100 ml (2, executed outside the plan)” when an item that is not indicated in the clinical pathway was executed. In this situation, the notation in the parentheses indicates (the date of execution or occurrence, a relationship with the clinical pathway). In this situation, the nominal scale may be divided into a plurality of levels. Further, two or more dates of execution or occurrence may collectively be converted into one label.

Further, by using each of the generated transactions, the extracting function 152 generates a correlation rule in which the data related to the health care action/outcome serves as a condition part, whereas the data related to the variance designated by the operator serves as a conclusion part and further calculates support, confidence, and lift values of the generated correlation rule. After that, the extracting function 152 generates correlation rule data in which the correlation rule is kept in correspondence with the index values and further stores the generated correlation rule data into the storage 120.

FIG. 7 is a table illustrating an example of the correlation rule data generated by the extracting function 152 according to the first embodiment.

For example, as illustrated in FIG. 7, the correlation rule data includes, as data items thereof, a clinical pathway code, a condition part, a conclusion part, a support value, a confidence value, and a lift value. In this situation, as the clinical pathway code, a code corresponding to the clinical pathway designated by the operator is set. Further, as the condition part, data related to the health care action/outcome is set. Further, as the conclusion part, data related to the variance designated by the operator is set. Further, as the support value, the confidence value, and the lift value, the values of the support, the confidence, and the lift calculated by the extracting function 152 are set, respectively.

In this situation, FIG. 7 illustrates the example of the correlation rule data that is generated when an association analysis is performed on the clinical pathway “colectomy/proctectomy (P0001)” and the variance “anastomotic leakage”. Further, the symbol “+” used in the condition part illustrated in FIG. 7 expresses a combination of health care actions or outcomes that occurred at the same time.

As explained above, in the correlation rule data, the conclusion part indicates the variance, whereas the condition part indicates a cause having correlation with the variance. Further, the support, the confidence, and the lift serve as correlation values each indicating a level of strength of the correlation between the cause and the variance.

Returning to the description of FIG. 1, the identifying function 153 is configured to identify a health care action relevant to a health care action causing a symptom of the patient subject to the health care, on the basis of the data related to the health care actions taken according to the clinical pathways and the data related to the variances occurring from the clinical pathway.

The first embodiment shall be explained while referring to the symptom of the patient subject to the health care as a “specific variance” and referring to the health care action causing the specific variance as a “cause subject to the analysis”, and referring to the health care action relevant to the cause subject to the analysis as a “relevant cause”.

On the basis of at least one of a plurality of axes indicating categories of descriptions of the health care actions, the identifying function 153 is configured to identify, as the relevant health care action, a health care action of which the description is similar to that of the health care action causing the symptom of the patient subject to the health care. In this situation, when the extracting function 152 has extracted data related to a plurality of patients, the identifying function 153 identifies the relevant health care action, on the basis of the data related to the health care actions taken for the plurality of patients and the data related to the symptoms thereof.

More specifically, via the input circuitry 130, the identifying function 153 receives an operation to designate a cause subject to the analysis, from the operator. After that, on the basis of the information extracted by the extracting function 152, the identifying function 153 identifies the relevant cause that is relevant to the cause subject to the analysis designated by the operator. In this situation, the relevant cause denotes a cause positioned close to the cause subject to the analysis, on at least one axis structured by information set in the correlation rule data. For example, the information structuring the axis in the present example may be information describing the health care action such as “the execution date of the health care action”, “the type of the health care action”, “attributes (age, gender, height, weight, etc.) of the patient for whom the health care action was taken”, and the like.

For example, from among the causes extracted by the extracting function 152, the identifying function 153 identifies, as the relevant cause, a cause of which the “execution date of the health care action” and the “type of the health care action” are similar to those of the cause subject to the analysis. In that situation, the identifying function 153 identifies the relevant cause by using two axes, namely, the “execution date of the health care action” and the “type of the health care action”.

More specifically, via the input circuitry 130, the identifying function 153 receives an operation to designate a range of time (a time span) related to the execution date, from the operator. After that, by referring to the correlation rule data, the identifying function 153 identifies one or more causes of which the execution item (e.g., Soldem 3A 500 ml) is the same as that of the cause subject to the analysis and of which only the time is different.

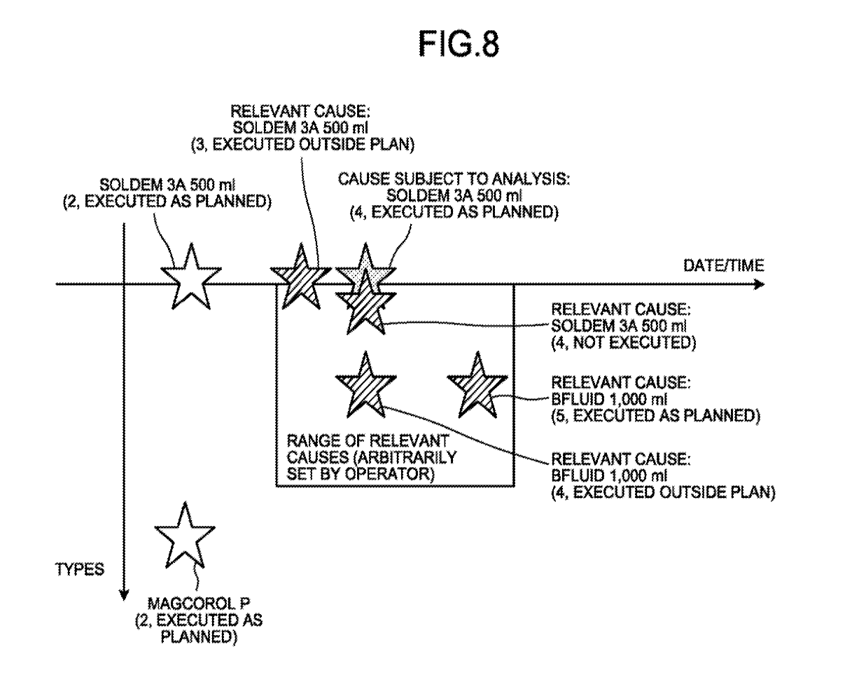

FIG. 8 is a drawing illustrating an example of the relevant cause identifying process performed by the identifying function 153 according to the first embodiment. In FIG. 8, the horizontal axis expresses the “execution date of the health care action” (date/time), whereas the vertical axis expresses the “type of the health care action” (types). Further, the star-shaped figures in FIG. 8 represent the causes extracted by the extracting function 152.

For example, as illustrated in FIG. 8, when the cause subject to the analysis is Soldem 3A 500 ml (4, executed as planned), the identifying function 153 identifies causes such as Soldem 3A 500 ml (2, executed as planned), Soldem 3A 500 ml (3, executed outside of the plan), Soldem 3A 500 ml (4, not executed), and the like. After that, from among the identified causes, the identifying function 153 further identifies one or more causes within the time span designated by the operator and determines the identified causes to be relevant causes. FIG. 8 illustrates an example in which Soldem 3A 500 ml (3, executed outside the plan) and Soldem 3A 500 ml (4, not executed) were identified according to the designated time span.

Further, by referring to the correlation rule data, the identifying function 153 identifies, as a relevant cause, a cause of which the parent execution item is the same as that of the execution item (e.g., Soldem 3A 500 ml) of the cause subject to the analysis. In this situation, for example, by referring to the execution item master data stored in the storage 120 in advance, the identifying function 153 identifies the cause of which the parent execution item is the same as that of the execution item of the cause subject to the analysis.

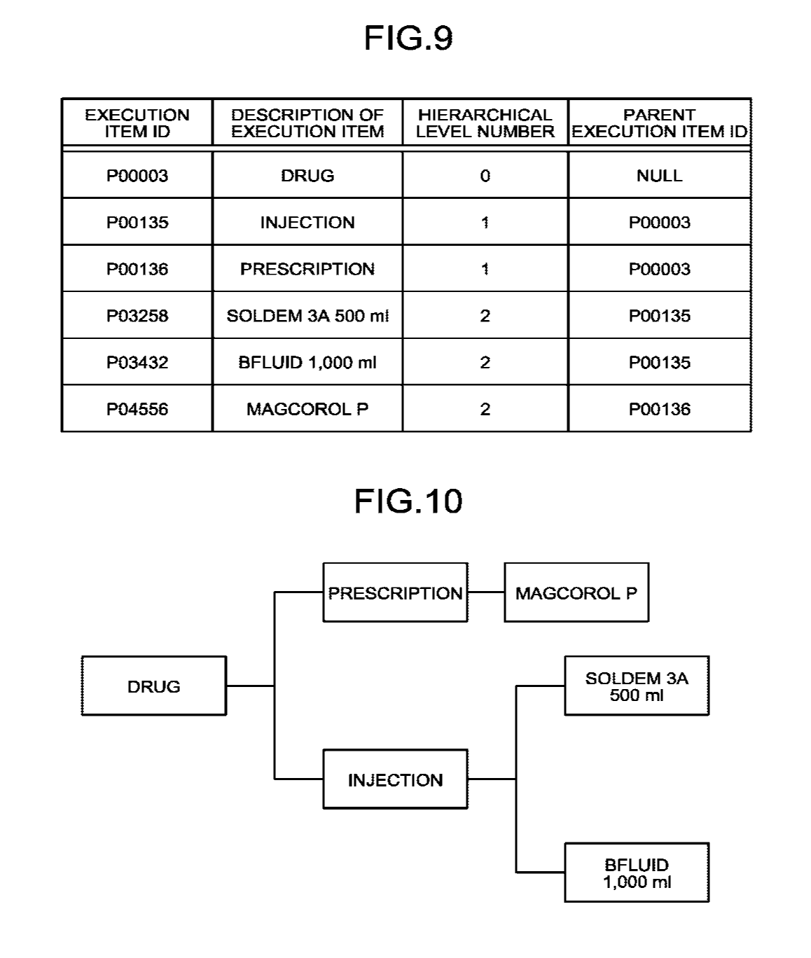

FIGS. 9 and 10 are drawings illustrating examples of the execution item master data used by the identifying function 153 according to the first embodiment.

For example, as illustrated in FIG. 9, the execution item master data includes, as data items thereof, an execution item ID, an execution item description, a hierarchical level number, and a parent execution item ID. In this situation, as the execution item ID, identification information uniquely identifying the execution item is set. Further, as the execution item description, information describing the execution item is set. Further, as the hierarchical level number, the hierarchical level number indicating the position of the execution item when the description of the execution item is expressed in a hierarchical manner is set. Further, as the parent execution item ID, identification information uniquely identifying the parent execution item (a superordinate execution item) of the execution item is set.

With respect to the example illustrated in FIG. 9, for example, as illustrated in FIG. 10, the item “drug” (execution item ID: P00003) serves as a parent execution item of “injection” (execution item ID: P00135) and “prescription” (execution item ID: P00136). Further, the item “injection” (execution item ID: P00135) serves as a parent execution item of “Soldem 3A 500 ml” (execution item ID: P03258) and “Bfluid 1,000 ml” (execution item ID: P03432). In addition, the item “prescription” (execution item ID: P00136) serves as a parent execution item of “Magcorol P” (execution item ID: P04556).

In the present example, for instance, as illustrated in FIG. 8, when the cause subject to the analysis is Soldem 3A 500 ml (4, executed as planned), the identifying function 153 extracts Bfluid 1,000 ml (5, executed as planned) and Bfluid 1,000 ml (4, executed outside the plan), and the like, of which the parent execution item ID is “P00135”. In the present example, because Bfluid 1,000 ml belongs to the parent execution item “injection”, like Soldem 3A 500 ml does, Bfluid 1,000 ml is identified as a relevant cause. In contrast, because Magcorol P belongs to the parent execution item “prescription” and not “injection”, Magcorol P is not identified as a relevant cause.

Further, the identifying function 153 may be configured to further identify one or more causes of which the parent execution item of the parent execution item is the same, in addition to identifying the one or more causes of which the parent execution item is the same as that of the execution item of the cause subject to the analysis. In that situation, for example, Magcorol P will further be identified, because the parent execution item of the parent execution item thereof is “drug” (execution item ID: P00003), like that of Soldem 3A 500 ml is. This type of condition related to the identifying process may arbitrarily be set by the operator, for example.

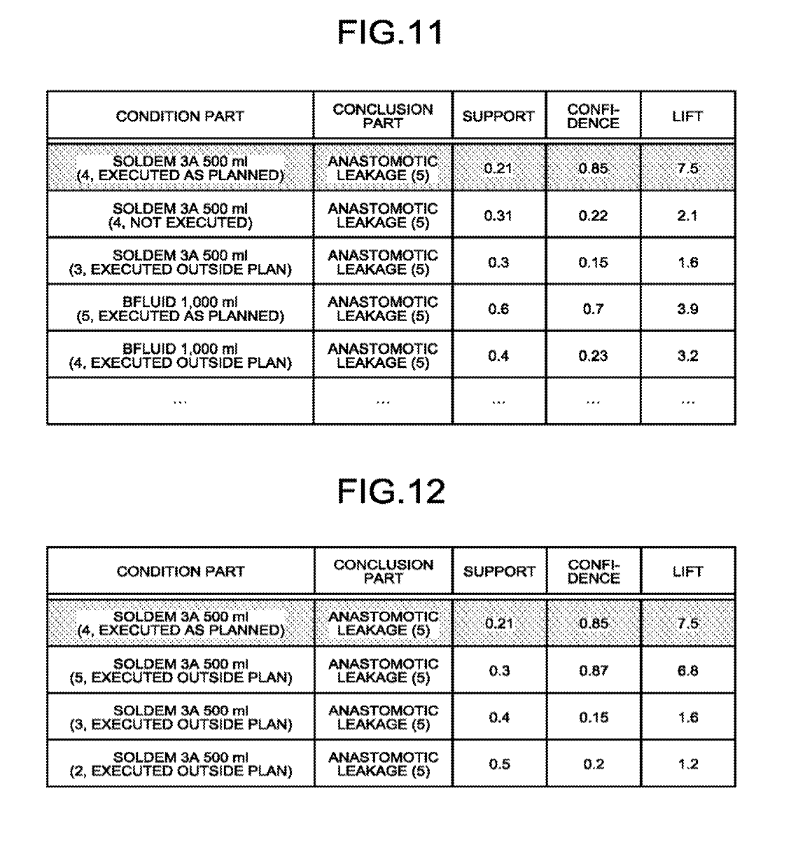

FIG. 11 is a table illustrating examples of the relevant causes identified by the identifying function 153 according to the first embodiment. The example in FIG. 11 illustrates the relevant causes that are identified when the cause subject to the analysis is “Soldem 3A 500 ml (4, executed as planned)”.

For example, as illustrated in FIG. 11, when the cause subject to the analysis is “Soldem 3A 500 ml (4, executed as planned)”, the identifying function 153 identifies the correlation rule data related to the present cause “Soldem 3A 500 ml (4, executed as planned)” as well as pieces of correlation rule data such as “Soldem 3A 500 ml (3, executed outside the plan)”, “Soldem 3A 500 ml (4, not executed)”, “Bfluid 1,000 ml (5, executed as planned)”, and “Bfluid 1,000 ml (4, executed outside the plan)”, and the like.

Returning to the description of FIG. 1, the predicting function 154 is configured to predict advantageous effects of candidates for an improvement plan, while using the relevant causes identified by the identifying function 153 as the candidates for the improvement plan.

More specifically, the predicting function 154 calculates, with respect to each of the candidates for the improvement plan, a change amount between a correlation value indicating the level of strength of correlation between the candidate and a specific variance and a correlation value indicating the level of strength of correlation between the cause subject to the analysis and the specific variance and further predicts an advantageous effect on the basis of the calculated change amount of the correlation values. For example, the predicting function 154 predicts the advantageous effect in such a manner that the larger the change amount of the correlation value is, the larger is the advantageous effect thereof.

In this situation, via the input circuitry 130, the predicting function 154 receives an operation to designate a factor which the operator wishes to improve, from the operator. After that, the predicting function 154 extracts necessary information from the relevant causes as the candidates for the improvement plan, in accordance with the factor which the operator wishes to improve that was designated by the operator and further compares the correlation between the cause subject to the analysis and the variance with the correlation between each of the candidates for the improvement plan and the variance. After that, the predicting function 154 predicts the advantageous effects in such a manner that the larger the change amount of the correlation value is, the lower is the degree of correlation between the candidate for the improvement plan and the variance, i.e., the larger is the advantageous effect of the improvement plan.

In the following sections, three examples corresponding to a factor which the operator wishes to improve will be explained, with respect to the predicting process performed by the predicting function 154 on the advantageous effects of the candidates for the improvement plan. In the present situation, an example will be explained in which the cause subject to the analysis is Soldem 3A 500 ml (4, executed as planned).

For example, when the factor the operator wishes to improve is execution timing of the cause subject to the analysis (a timing change), the predicting function 154 extracts, as the “candidates for the improvement plan”, one or more causes related to the “timing change” from among the relevant causes. More specifically, the predicting function 154 extracts one or more of the relevant causes related to the “timing change”, by using an extracting condition “having the same execution item name (Soldem 3A 500 ml) & having a different execution date & being executed outside the plan”. After that, the predicting function 154 calculates a change amount of the correlation value by comparing the correlation value of at least one candidate for the improvement plan that was extracted with the correlation value of the cause subject to the analysis.

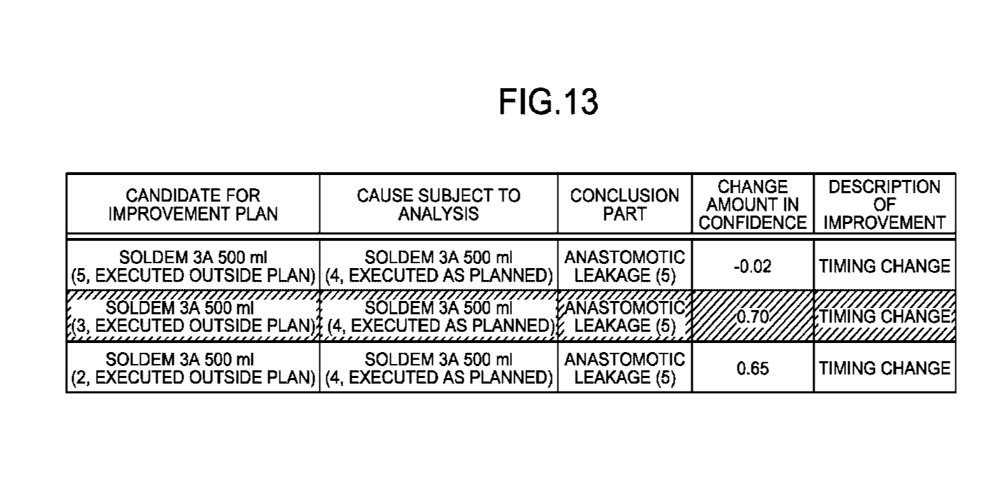

FIGS. 12 and 13 are tables illustrating examples of the advantageous effect predicting process performed by the predicting function 154 according to the first embodiment on the candidates for the improvement plan related to the timing change.

For example, as illustrated in FIG. 12, when the cause subject to the analysis is “Soldem 3A 500 ml (4, executed as planned)”, the predicting function 154 extracts, as candidates for the improvement plan, data related to “Soldem 3A 500 ml (5, executed outside the plan)”, data related to “Soldem 3A 500 ml (3, executed outside the plan)”, and data related to “Soldem 3A 500 ml (2, executed outside the plan)”.

After that, for example, as illustrated in FIG. 13, with respect to each of the extracted candidates for the improvement plan, the predicting function 154 calculates a change amount in the confidence value by comparing the confidence value thereof with the confidence value of “Soldem 3A 500 ml (4, executed as planned)” serving as a cause subject to the analysis. Further, the predicting function 154 predicts the candidate having the largest change amount in the confidence value among the candidates for the improvement plan to be an improvement plan having the largest advantageous effect. In other words, in the example illustrated in FIG. 13, the predicting function 154 predicts “Soldem 3A 500 ml (3, executed outside the plan)” having the largest change amount “0.70” in the confidence value among the three candidates for the improvement plan, to be an improvement plan having the largest advantageous effect.

In another example, when the factor the operator wishes to improve is the type of the cause subject to the analysis (a type change), the predicting function 154 extracts, as the “candidates for the improvement plan”, one or more causes related to the “type change” from among the relevant causes. In that situation, the predicting function 154 extracts the one or more of the relevant causes related to the “type change”, by using an extracting condition “having a different execution item name & having the same execution date & being executed outside the plan”. After that, the predicting function 154 calculates a change amount of the correlation value by comparing the correlation value of at least one candidate for the improvement plan that was extracted with the correlation value of the cause subject to the analysis.

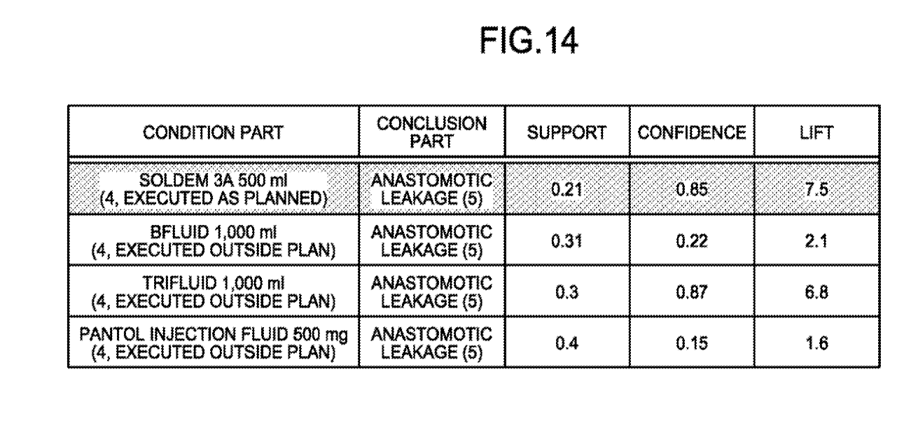

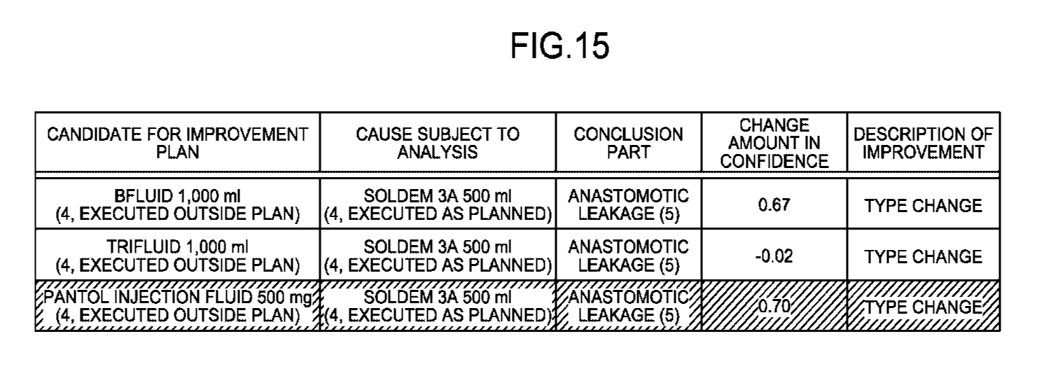

FIGS. 14 and 15 are tables illustrating an example of the advantageous effect predicting process performed by the predicting function 154 according to the first embodiment on the candidates for the improvement plan related to the type change.

For example, as illustrated in FIG. 14, when the cause subject to the analysis is “Soldem 3A 500 ml (4, executed as planned)”, the predicting function 154 extracts, as candidates for the improvement plan, data related to “Bfluid 1,000 ml (4, executed outside the plan)”, data related to “Trifluid 1,000 ml (4, executed outside the plan)”, and data related to “Pantol injection fluid 500 mg (4, executed outside the plan)”.

After that, for example, as illustrated in FIG. 15, with respect to each of the extracted candidates for the improvement plan, the predicting function 154 calculates a change amount in the confidence value by comparing the confidence value thereof with the confidence value of “Soldem 3A 500 ml (4, executed as planned)” serving as a cause subject to the analysis. Further, the predicting function 154 predicts the candidate having the largest change amount in the confidence value among the candidates for the improvement plan to be an improvement plan having the largest advantageous effect. In other words, in the example illustrated in FIG. 15, the predicting function 154 predicts “Pantol injection fluid 500 mg (4, executed outside the plan)” having the largest change amount “0.70” in the confidence value among the three candidates for the improvement plan, to be an improvement plan having the largest advantageous effect.

In yet another example, when the factor the operator wishes to improve is execution/non-execution of the cause subject to the analysis (a change between execution/non-execution), the predicting function 154 extracts, as the “candidates for the improvement plan”, one or more causes related to the “change between execution/non-execution” from among the relevant causes. In that situation, the predicting function 154 extracts the one or more of the relevant causes related to the “change between execution/non-execution”, by using an extracting condition “having the same execution item name & having the same execution date & not being executed”. After that, the predicting function 154 calculates a change amount of the correlation value by comparing the correlation value of at least one candidate for the improvement plan that was extracted with the correlation value of the cause subject to the analysis.

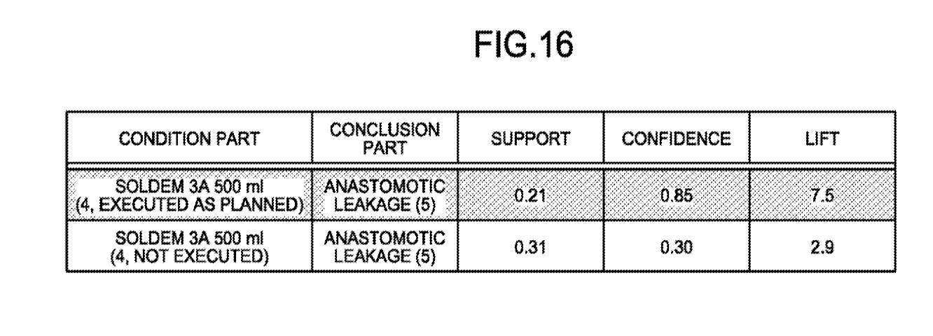

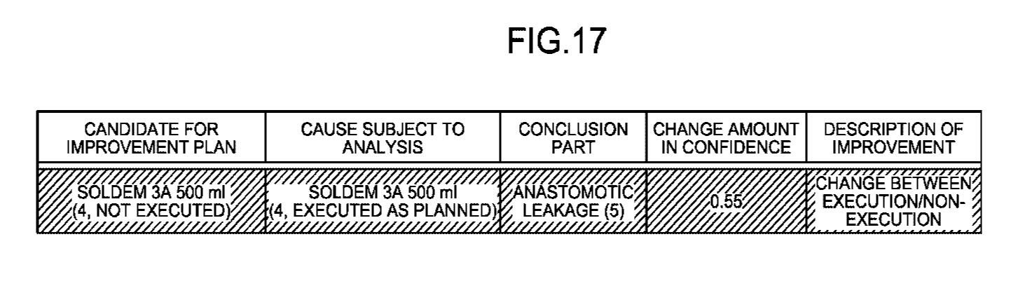

FIGS. 16 and 17 are tables illustrating examples of the advantageous effect predicting process performed by the predicting function 154 according to the first embodiment on the candidates for the improvement plan related to the change between execution/non-execution.

For example, as illustrated in FIG. 16, when the cause subject to the analysis is “Soldem 3A 500 ml (4, executed as planned)”, the predicting function 154 extracts data related to “Soldem 3A 500 ml (4, not executed)” as a candidate for the improvement plan.

After that, for example, as illustrated in FIG. 17, with respect to each of the extracted candidates for the improvement plan, the predicting function 154 calculates a change amount in the confidence value by comparing the confidence value thereof with the confidence value of “Soldem 3A 500 ml (4, executed as planned)” serving as a cause subject to the analysis. Further, the predicting function 154 predicts the candidate having the largest change amount in the confidence value among the candidates for the improvement plan to be an improvement plan having the largest advantageous effect. In this situation, in the example illustrated in FIG. 17, because there is one candidate for the improvement plan, the predicting function 154 predicts “Soldem 3A 500 ml (4, not executed)” having the change amount “0.55” in the confidence value, to be an improvement plan having the largest advantageous effect.

In the above sections, the examples are explained in which the predicting function 154 uses the “timing change”, the “type change”, or the “change between execution/non-execution” as the factor the operator wishes to improve; however, possible embodiments are not limited to these examples. For instance, the predicting function 154 may predict advantageous effects of the candidates for the improvement plan by combining together two or more factors which the operator wishes to improve, such as “a timing change and a type change”.

Further, in the above sections, the examples are explained in which the predicting function 154 uses the confidence values as the correlation values; however, possible embodiments are not limited to these examples. For instance, the predicting function 154 may predict advantageous effects of the candidates for the improvement plan by using either the support values or the lift values as the correlation values.

Returning to the description of FIG. 1, the display controlling function 155 is configured to cause the display 140 to display, with respect to each of the candidates for the improvement plan, information indicating the advantageous effect thereof predicted by the predicting function 154.

More specifically, with respect to the clinical pathway, the variance, and the cause subject to the analysis that were designated by the operator, the display controlling function 155 generates a screen presenting the candidates for the improvement plan and information indicating the advantageous effects of the candidates for the improvement plan and further causes the display 140 to display the generated screen.

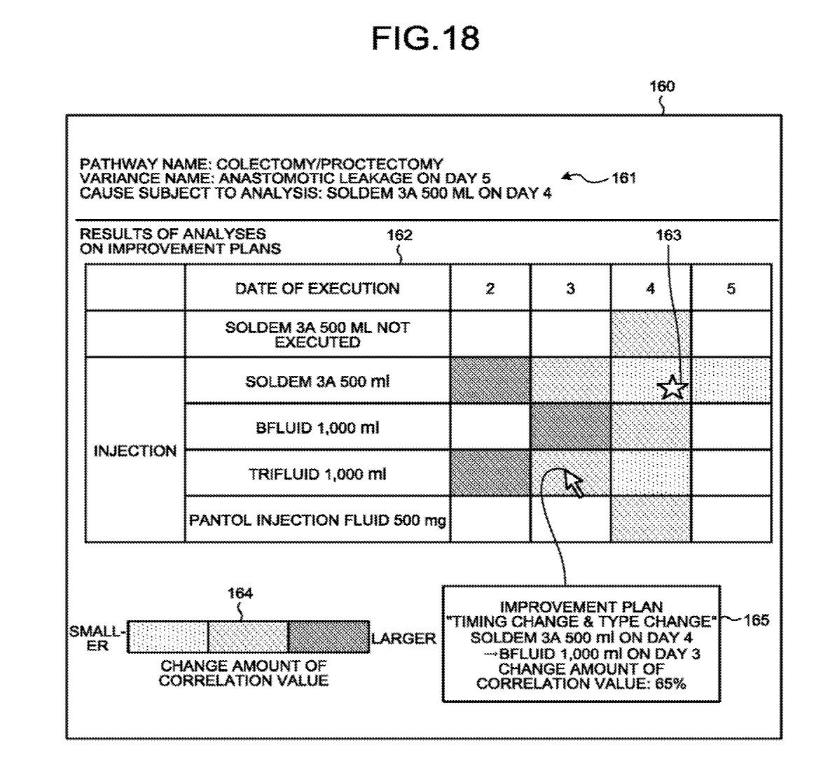

FIG. 18 is a drawing illustrating an example of the screen displayed by the display controlling function 155 according to the first embodiment.

For example, as illustrated in FIG. 18, the display controlling function 155 generates a screen 160 having arranged therein information 161 that indicates the pathway name of a clinical pathway, the name of a variance, and a cause subject to an analysis as well as a table 162 indicating candidates for the improvement plan and further causes the display 140 to display the generated screen 160.

For example, as the table 162, the display controlling function 155 displays a table indicating each of the plurality of candidates for the improvement plan as a set made up of an execution date and a type, so that the execution dates of the improvement plan are indicated in a time-series order in the horizontal direction, while the types of the improvement plan are indicated in the vertical direction. Further, for example, in the table 162, the display controlling function 155 displays a mark 163 represented by a predetermined figure (a star in the example in FIG. 18) in the section corresponding to the cause subject to the analysis. In this manner, because the display controlling function 155 displays the plurality of candidates for the improvement plan in the time series and for each of the types, it is possible to easily understand the correspondence relationship with the clinical pathway.

Further, with respect to each of the plurality of candidates for the improvement plan, the display controlling function 155 displays, in a corresponding section within the table 162, information indicating the magnitude of the advantageous effect of the candidate for the improvement plan. More specifically, on the basis of the magnitude of the change amounts of the correlation values calculated by the predicting function 154, the display controlling function 155 displays the information indicating the magnitude of the advantageous effect of each of the candidates for the improvement plan. For example, in accordance with the magnitude of each of the change amounts of the correlation values, the display controlling function 155 displays the sections in the table 162 by using colors having mutually-different levels of darkness. More specifically, for example, the display controlling function 155 arranges the colors of the sections in the table 162 in such a manner that the larger the change amount of the correlation value is, the darker is the color of the section. In this situation, for such sections that have no corresponding candidate for the improvement plan, the display controlling function 155 displays the sections without any color. In that situation, for example, the display controlling function 155 displays, on the screen 160, a bar-shaped graphic element 164 indicating the correspondence relationship between the magnitude of the change amounts of the correlation values and the levels of darkness of the colors. In this manner, because the display controlling function 155 displays, in the table 162, the magnitude of the change amount of the correlation value with respect to each of the candidates for the improvement plan by using the levels of darkness of the colors, the operator is able to easily understand the improvement plans having larger change amounts of correlation value, i.e., the improvement plans having larger advantageous effects.

Further, by receiving, from the operator, an operation to select one of the plurality of sections of the table 162, the display controlling function 155 receives, from the operator, an operation to select one of the plurality of candidates for the improvement plan. After that, when the one of the candidates for the improvement plan has been selected by the operator, the display controlling function 155 displays, on the screen 160, information 165 indicating a specific description of the improvement and advantageous effects thereof, with respect to the selected candidate for the improvement plan. In this situation, as the information indicating the advantageous effects of the candidate for the improvement plan, the display controlling function 155 displays the magnitude of the change amount of the correlation value. In this manner, as a result of the display controlling function 155 displaying, on the screen 160, the information 165 indicating the specific description of the improvement and the advantageous effects thereof with respect to the candidate for the improvement plan selected by the operator out of the table 162, the operator is able to easily check, on the screen 160, the specific description of the improvement and the advantageous effects thereof with respect to each of the candidates for the improvement plan.

Processing functions of the processing circuitry 150 have thus been explained. The processing functions described above are stored in the storage 120 in the form of computer-executable programs, for example. The processing circuitry 150 realizes the processing functions corresponding to the programs by reading the programs from the storage 120 and executing the read programs. In other words, the processing circuitry 150 that has read the programs has the processing functions illustrated in FIG. 1.

Although FIG. 1 illustrates the example in which the processing functions described above are realized only by the processing circuitry 150, possible embodiments are not limited to this example. For instance, the processing circuitry 150 may be structured by combining together a plurality of independent processors, so that the processors realize the processing functions by executing the programs. Further, any of the processing functions of the processing circuitry 150 may be realized as being distributed to a plurality of processing circuits or being integrated into a single processing circuit, as appropriate.

Further, the term “processor” used in the above explanations denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The processors each realize the functions thereof by reading and executing the program saved in the storage 120. In this situation, instead of saving the programs in the storage 120, it is also acceptable to directly incorporate the programs in the circuits of the processors. In that situation, the processors realize the functions thereof by reading and executing the programs incorporated in the circuits thereof. Further, the processors in the present embodiments do not each necessarily have to be structured as a single circuit. It is also acceptable to structure one processor by combining together a plurality of independent circuits so as to realize the functions thereof.

In this situation, the programs executed by the processors are provided as being incorporated, in advance, into a Read-Only Memory (ROM), a storage, or the like. Alternatively, the programs may be provided for those devices as being recorded on a computer-readable storage medium such as a Compact Disk Read-Only Memory (CD-ROM), a flexible disk (FD), a Compact Disk Recordable (CD-R), a Digital Versatile Disk (DVD), or the like, in a file that is in an installable format or in an executable format. Further, the programs may be stored in a computer connected to a network such as the Internet, so as to be provided or distributed as being downloaded via the network. For example, each of the programs is structured with a module including functional units described later. In actual hardware, as a result of a CPU reading and executing the programs from a storage medium such as a ROM, the modules are loaded into a main storage device so as to be generated in the main storage device.

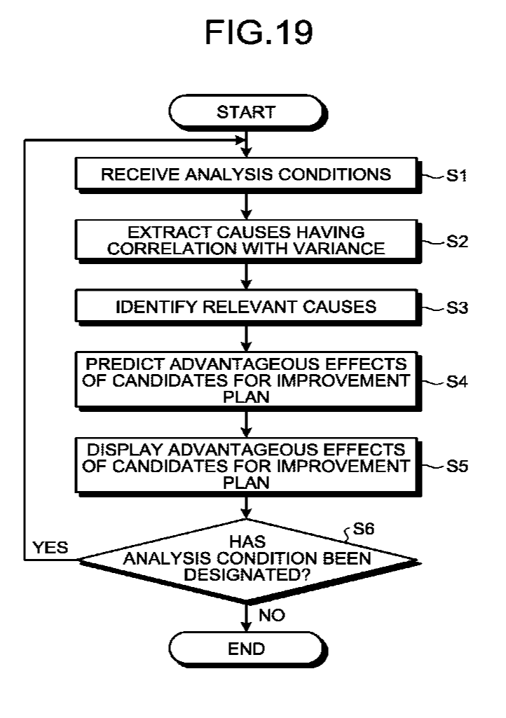

FIG. 19 is a flowchart illustrating a processing procedure in a process performed by the medical information processing apparatus 100 according to the first embodiment. It should be noted that the process performed by the obtaining function 151 to obtain the data related to the health care actions taken according to the clinical pathways and the data related to the variances occurring from the clinical pathways is performed not in synchronization with the processing procedure explained below. In this situation, the process performed by the obtaining function 151 is, for example, realized as a result of the processing circuitry 150 reading and executing a predetermined program corresponding to the obtaining function 151 from the storage 120.

For example, as illustrated in FIG. 19, in the present embodiment, the extracting function 152 receives analysis conditions (a clinical pathway and a variance) from the operator (step S1). After that, the extracting function 152 extracts causes each having correlation with the variance designated by the operator, on the basis of the data related to the health care actions taken according to the clinical pathway designated by the operator and the data related to the variances occurring from the clinical pathways (step S2).

Subsequently, the identifying function 153 identifies relevant causes that are relevant to a cause subject to an analysis, from among the causes extracted by the extracting function 152 (step S3).

Subsequently, while using the relevant causes identified by the identifying function 153 as candidates for an improvement plan, the predicting function 154 predicts advantageous effects of each of the candidates for the improvement plan (step S4).

After that, the display controlling function 155 causes the display 140 to display information indicating the advantageous effect predicted by the predicting function 154 with respect to each of the candidates for the improvement plan (step S5).

In this situation, when a new analysis condition is designated by the operator (step S6: Yes), the process returns to step S1 so that the processing procedure described above is performed again. On the contrary, when no analysis condition is designated by the operator (step S6: No), the process is ended.

Steps S1 and S2 described above are realized, for example, as a result of the processing circuitry 150 reading and executing a predetermined program corresponding to the extracting function 152 from the storage 120. Step S3 is realized, for example, as a result of the processing circuitry 150 reading and executing a predetermined program corresponding to the identifying function 153 from the storage 120. Step S4 is realized, for example, as a result of the processing circuitry 150 reading and executing a predetermined program corresponding to the predicting function 154 from the storage 120. Step S5 is realized, for example, as a result of the processing circuitry 150 reading and executing a predetermined program corresponding to the display controlling function 155 from the storage 120.

As explained above, in the first embodiment, the identifying function 153 is configured to identify the relevant causes that are relevant to the cause subject to the analysis, on the basis of the data related to the health care actions taken according to the clinical pathways and the data related to the variances occurring from the clinical pathways. Further, the predicting function 154 is configured to predict the advantageous effects of each of the candidates for the improvement plan, while using the relevant causes identified by the identifying function 153 as the candidates for the improvement plan. Consequently, according to the first embodiment, it is possible to present the effective improvement plan related to the clinical pathways.

For example, according to some conventional techniques, improvement items for a clinical pathway are extracted and presented on the basis of data related to variances; however, when such improvement items are simply presented, it is difficult, with respect to improvement plans, which improvement plan is effective when being executed. For example, when “administering an antibiotic” is presented as an improvement item, the user himself/herself will have to determine whether or not the administration of the antibiotic should be stopped, whether or not the type of the antibiotic should be changed, and whether or not the timing with which the antibiotic is administered should be changed. In contrast to such conventional techniques, because the effective improvement plan related to the clinical pathway is presented according to the present embodiment described above, the user is able to easily determine an appropriate improvement plan.

### Second Embodiment

In the embodiment described above, the example is explained in which the identifying function 153 is configured to identify the relevant causes that are relevant to the cause subject to the analysis on the basis of the range designated by the operator; however, possible embodiments are not limited to this example.

In the following sections, as a second embodiment, an example will be explained in which the identifying function 153 is configured to set a condition used for identifying relevant causes that are relevant to a cause subject to an analysis, on the basis of at least one selected from between the quantity and a distribution of the causes extracted by the extracting function 152. The second embodiment will be explained while a focus is placed on differences from the embodiment described above. Explanations of elements that are duplicate of those in the above embodiment will be omitted.

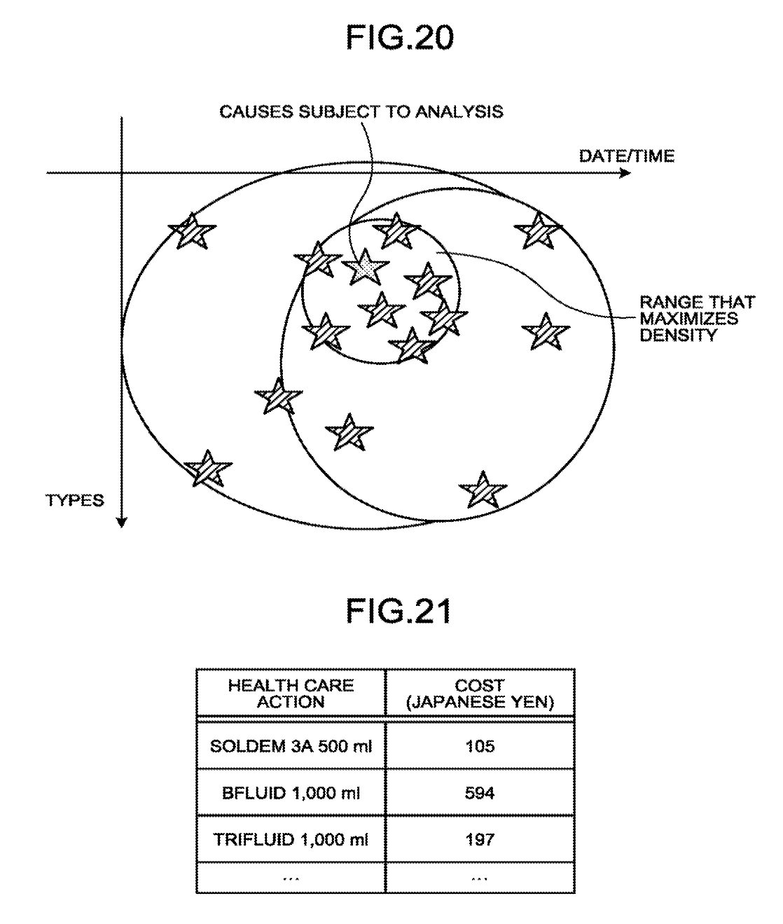

FIG. 20 is a drawing illustrating an example of the relevant cause identifying process performed by the identifying function 153 according to the second embodiment. FIG. 20 illustrates an example in which, similarly to the example in FIG. 8, the horizontal axis expresses the “execution date of the health care action” (date/time) whereas the vertical axis expresses the “type of the health care action” (types). Further, similarly to the example in FIG. 8, the star-shaped figures in FIG. 20 represent the causes extracted by the extracting function 152.

For example, as illustrated in FIG. 20, when the data of the causes extracted by the extracting function 152 is arranged in a coordinate system in which the “execution date of the health care action” (date/time) is expressed on the horizontal axis, whereas the “type of the health care action” (types) is expressed on the vertical axis, the identifying function 153 sets such a range that includes the data of the cause subject to the analysis and pieces of data in the surroundings thereof and that maximizes the density of the data. Further, on the basis of the set range, the identifying function 153 identifies relevant causes that are relevant to the cause subject to the analysis. More specifically, in that situation, the identifying function 153 identifies the causes that are in the range set on the basis of the density of the data, from among the causes extracted by the extracting function 152 and thus uses the identified causes as the relevant causes.

In this manner, in the second embodiment, the identifying function 153 is configured to set the condition used for identifying the relevant causes, on the basis of at least one selected from between the quantity and the distribution of the causes extracted by the extracting function 152. Consequently, according to the second embodiment, it is possible to arrange the condition used for identifying the relevant causes to be an optimal condition in accordance with the quantity or the distribution of the causes. It is therefore possible to effectively extract the causes that are closely relevant to the cause subject to the analysis.

### Third Embodiment

In the embodiments described above, the example is explained in which the predicting function 154 is configured to predict the advantageous effects on the basis of the change amounts of the correlation values each indicating the level of strength of correlation with the variance, with respect to each of the candidates for the improvement plan; however, possible embodiments are not limited to this example.

In the following sections, as a third embodiment, an example will be explained in which the predicting function 154 is configured to further calculate, for each of the candidates for the improvement plan, a change amount between a cost related to the candidate and a cost related to the cause subject to an analysis and to predict advantageous effects on the basis of the calculated change amounts in the cost and the change amounts of the correlation values. For example, the predicting function 154 predicts the advantageous effects in such a manner that the smaller the change amount is, the larger is the advantageous effect when the change amount in the cost is a positive value and that the larger the change amount is, the larger is the advantageous effect when the change amount in the cost is a negative value. The third embodiment will be explained while a focus is placed on differences from the embodiments described above. Explanations of elements that are duplicate of those in the above embodiments will be omitted.

For example, by referring to cost data stored in the storage 120 in advance, the predicting function 154 obtains a cost related to the cause subject to the analysis and a cost related to each of the candidates for the improvement plan. Further, for each of the candidates for the improvement plan, the predicting function 154 calculates a change amount between the cost related to the candidate and the cost related to the cause subject to the analysis and further predicts the advantageous effect of each of the candidates for the improvement plan, on the basis of the calculated change amount in the cost and the change amount of the correlation value described in the embodiments above.

FIG. 21 is a table illustrating an example of the cost data used by the predicting function 154 according to the third embodiment.

For example, as illustrated in FIG. 21, the cost data includes, as data items thereof, a health care action and a cost (Japanese Yen) thereof. In this situation, as the health care action, information indicating a health care action taken for the patient is set. Further, as the cost (Japanese Yen), a price (Japanese Yen) indicating the cost of the health care action is set. Alternatively, for example, instead of the price, medical remuneration points may be set as the cost.

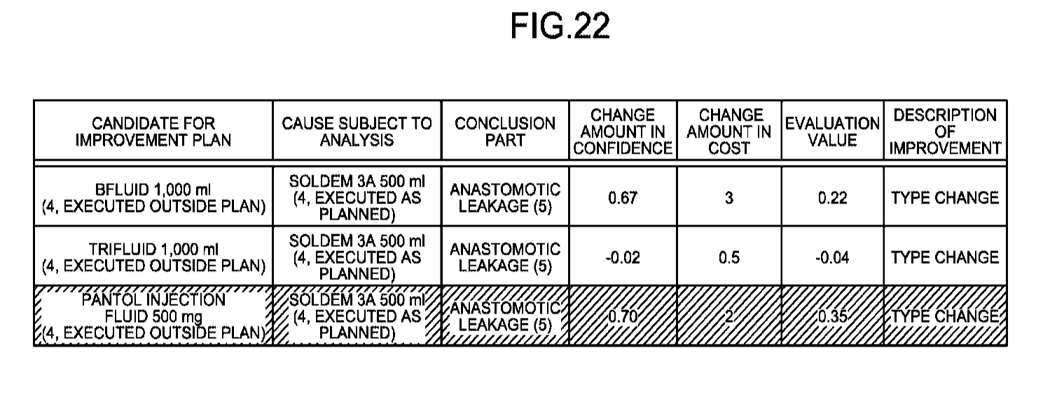

FIG. 22 is a table illustrating an example of the advantageous effect predicting process performed by the predicting function 154 according to the third embodiment on candidates for an improvement plan.

For example, as illustrated in FIG. 22, the predicting function 154 calculates, for each of the candidates for the improvement plan, a change amount in the cost by comparing the cost thereof with the cost of the cause subject to the analysis. In this situation, for example, as the change amount in the cost, the predicting function 154 calculates how much more (e.g., how many times as much, etc.) the cost related to each of the candidates for the improvement is, compared to the cost related to the cause subject to the analysis.

Further, with respect to each of the candidates for the improvement plan, the predicting function 154 calculates a value expressed as “the change amount in the confidence value×(1/the change amount in the cost)” as an evaluation value when the change amount in the cost is a positive value and calculates a value expressed as “the change amount in the confidence value×|the change amount in the cost|” as an evaluation value when the change amount in the cost is a negative value. Further, the predicting function 154 predicts one of the candidates for the improvement plan having the largest evaluation value to be an improvement plan having the largest advantageous effect. In other words, in the example illustrated in FIG. 22, the predicting function 154 predicts “Pantol injection fluid 500 mg (4, executed outside the plan)” of which the evaluation value “0.35” is the largest among the three candidates for the improvement plan to be an improvement plan having the largest advantageous effect.

After that, in the third embodiment, with respect to each of the plurality of candidates for the improvement plan, the display controlling function 155 displays, instead of the magnitude of the change amount of the correlation value, information indicating the magnitude of the advantageous effect of the candidate for the improvement plan on the basis of the magnitude of the evaluation value thereof.

As explained above, in the third embodiment, the predicting function 154 is configured to predict the advantageous effect of each of the candidates for the improvement plan, on the basis of both the change amount of the correlation value with the variance and the change amount in the cost. Consequently, according to the third embodiment, it is possible to present a more effective improvement plan that also takes the costs into consideration.

According to at least one aspect of the embodiments described above, it is possible to present the effective improvement plan related to the health care actions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuitry configured to:
obtain data related to health care actions and data related to symptoms of a subject occurring from the health care actions; and
identify a health care action relevant to a health care action causing a symptom of the subject, on a basis of the data related to the health care actions and the data related to the symptoms.

2. The medical information processing apparatus according to claim 1, wherein
the processing circuitry further extracts correlation information indicating a level of strength of correlation between the symptom of the subject and the health care action causing the symptom, on the basis of the data related to the health care actions and the data related to the symptoms, and
the processing circuitry identifies the relevant health care action on a basis of the extracted correlation information.

3. The medical information processing apparatus according to claim 1, wherein, on a basis of at least one of a plurality of axes indicating categories of descriptions of the health care actions, the processing circuitry identifies, as the relevant health care action, a health care action of which description is similar to that of the health care action causing the symptom of the subject.

4. The medical information processing apparatus according to claim 3, wherein the description of the health care action denotes at least one selected from among: an execution date of the health care action; a type of the health care action; and an attribute of the subject for whom the health care action was taken.

5. The medical information processing apparatus according to claim 3, wherein the processing circuitry sets a condition used for identifying the relevant health care action, on a basis of at least one selected from between a quantity and a distribution of health care actions causing the symptom of the subject.

6. The medical information processing apparatus according to claim 1, wherein the processing circuitry identifies the relevant health care action, on a basis of data related to health care actions taken for a plurality of subjects and data related to the symptoms thereof.

7. The medical information processing apparatus according to claim 1, wherein, by using the relevant health care action as a candidate for an improvement plan, the processing circuitry further predicts an advantageous effect of the candidate for the improvement plan.

8. The medical information processing apparatus according to claim 7, wherein, with respect to each of candidates for the improvement plan, the processing circuitry calculates a change amount between a correlation value indicating a level of strength of correlation between the candidate and the symptom of the subject and a correlation value indicating a level of strength of correlation between the health care action causing the symptom of the subject and the symptom of the subject and predicts the advantageous effect on a basis of the calculated change amounts of the correlation values.

9. The medical information processing apparatus according to claim 8, wherein the processing circuitry predicts the advantageous effect in such a manner that the larger the change amount of the correlation value is, the larger is the advantageous effect.

10. The medical information processing apparatus according to claim 8, wherein, with respect to each of the candidates for the improvement plan, the processing circuitry further calculates a change amount between a cost related to the candidate and a cost related to the health care action causing the symptom of the subject and predicts the advantageous effect on a basis of the change amount in the cost and the change amount of the correlation value that were calculated.

11. The medical information processing apparatus according to claim 10, wherein
the processing circuitry predicts the advantageous effect in such a manner that the smaller the change amount is, the larger is the advantageous effect, when the change amount in the cost is a positive value, and
the processing circuitry predicts the advantageous effect in such a manner that the larger the change amount is, the larger is the advantageous effect, when the change amount in the cost is a negative value.

12. The medical information processing apparatus according to claim 7, wherein, with respect to each of candidates for the improvement plan, the processing circuitry further causes a display to display information indicating the advantageous effect thereof.

13. The medical information processing apparatus according to claim 1, wherein
the data related to the health care actions is data related to health care actions in a clinical pathway,
the data related to the symptoms is data related to variances, and
the processing circuitry identifies a health care action relevant to a health care action causing a variance for the subject.

14. A medical information processing apparatus comprising a processing circuitry configured to:
obtain data related to health care actions in a clinical pathway and data related to variances for a subject occurring from the health care actions; and
identify a health care action relevant to a health care action causing a variance for the subject, on a basis of the data related to the health care actions and the data related to the variances.

15. A medical information processing method comprising:
obtaining data related to health care actions and data related to symptoms of a subject occurring from the health care actions; and
identifying a health care action relevant to a health care action causing a symptom of the subject, on a basis of the data related to the health care actions and the data related to the symptoms.
